(19) United States
(12) Patent Application Publication
(10) Pub. No.: US 2007/0233190 A1
(43) Pub. Date: Oct. 4, 2007

(54) METHOD AND APPARATUS FOR VERTEBRAE REALIGNMENT THROUGH MUSCLE RELAXATION AND SPINAL MANIPULATION

(76) Inventors: Philip A. Forsey, Henderson, NV (US); Chris Johnson, Lewiston, ID (US)

Correspondence Address: DYKAS, SHAVER & NIPPER, LLP, P.O. BOX 877, BOISE, ID, 83701-0877, US

(21) Appl. No.: 11/680,064

(22) Filed: Feb. 28, 2007

Related U.S. Application Data
(60) Provisional application No. 60/767,053, filed on Feb. 28, 2006.

(51) Int. Cl. A61F 5/00

(52) U.S. Cl. 606/237

(57) ABSTRACT

A method and apparatus for vertebrae realignment through muscle relaxation and spinal manipulation, using a pillow-shaped body portion having an adjustable manipulation device for exerting pressure on the spine, heating device for providing heat, and a vibration device for providing vibration, all cooperating to manipulate the user's spine into proper alignment.

### CROSS-REFERENCE TO RELATED APPLICATIONS

This application is a non-provisional of application Ser. No. 60/767053 entitled Device for Vertebrae Re-Alignment through Muscle Relaxation & Spinal Manipulation, filed by Philip A. Forsey and Chris Johnson, on Feb. 28, 2006, the disclosure of which is incorporated herein by this reference.

### FIELD OF THE INVENTION

The present invention generally relates to orthopedic devices, and more specifically relates to the utilization of a pillow or other aid to relax a user's spine and allow the manipulation of a user's spinal column back into correct alignment.

### BRIEF DESCRIPTION OF THE DRAWINGS

FIG. 1 is a front side perspective view of one embodiment of the present invention.

FIG. 2 is a cross-sectional view of the embodiment of FIG. 1.

FIG. 3 is a partial, side view of one embodiment of an alignment cell of the present invention as used in the embodiment of FIG. 1.

FIG. 4 is a front side perspective view of a second embodiment of the present invention.

FIG. 5 is a cross-sectional view of the embodiment of FIG. 4.

FIG. 6 is a partial, side view of one embodiment of a mechanical manipulation device of the present invention.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

In the following description and in the figures, like elements are identified with like reference numerals. The use of “or” indicates a non-exclusive alternative without limitation unless otherwise noted. The use of “including” means “including, but not limited to,” unless otherwise noted.

The present invention is a method and apparatus for vertebrae realignment through muscle relaxation and spinal manipulation. While the disclosure of the invention focuses upon a couple of the apparatus embodiments, other apparatus embodiments, as well as the method, also make up the present invention.

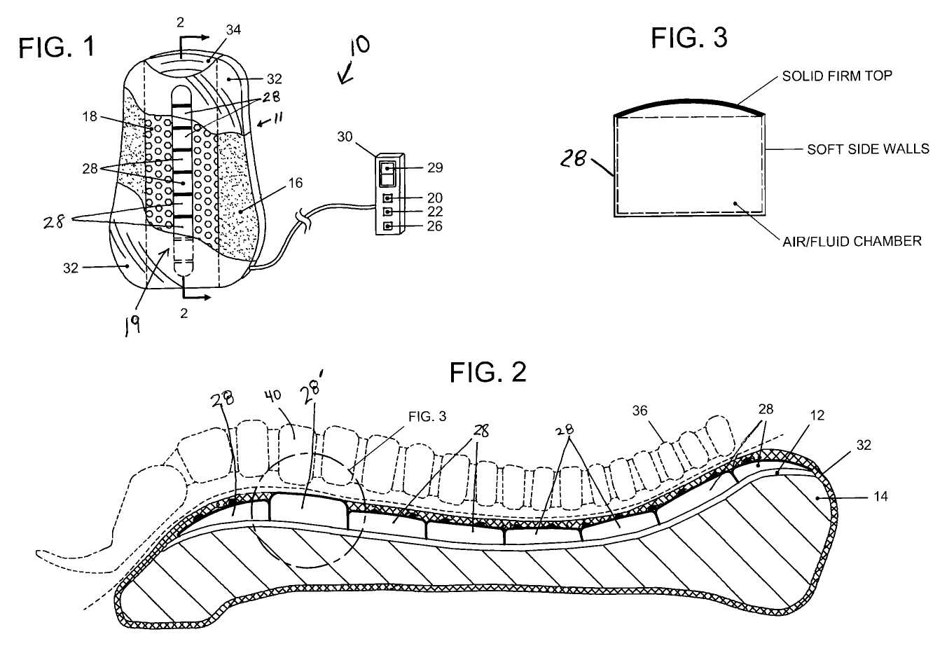

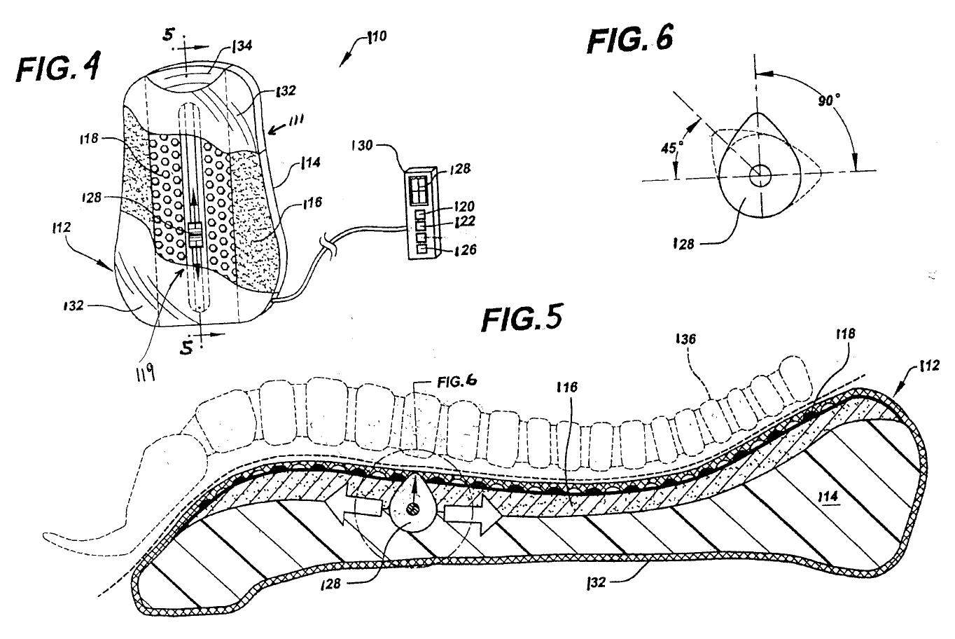

A first embodiment (device for manipulating the spine) 10 of the present invention is shown in FIGS. 1-3. A second embodiment (device for manipulating the spine) 110 of the present invention is shown in FIGS. 4-6. The first embodiment showing a pneumatic/hydraulic manipulation device, whereas the second embodiment shows a mechanical manipulation device.

Referring initially to FIGS. 1-3, this embodiment of the present invention comprising a body portion 11 (pillow) contoured to generally correspond to the natural curve (lower, middle, and upper areas of the spine (lumbar, thoracic, cervical) as well as the head and neck) of a human spine 36. The body portion 11 defining a medial axis (generally along which the cross-section of FIG. 2 is taken) which is configured for generally aligning parallel to the spine of the user in use.

In preferred use, a user would place the device 10 upon a surface (preferably firm), such as the floor or a bed, and would lay supine upon the device, generally aligning his/her spine with the contours of the body portion 11. It is preferred that the body portion be cushioned, having a solid (firm) bottom portion (base unit) 14, a soft contoured top 16, and being covered with a cover or covering.

This embodiment comprising a manipulation device 19 for selectively applying pressure against a selected region of the user's spine, preferably on a particular vertebrae and/or the vertebrae adjacent thereto, for assisting in manipulating the spine back into correct alignment. In the embodiment shown, the manipulation device comprises a plurality of selectively inflatable bladders (alignment cells) 28. The preferred alignment cells, particularly shown in FIG. 3, are individual cells, having an air/fluid chamber therein, soft sidewalls and a solid/firm top. By adjusting the pressure of the fluid (such as air) held therein, the firmness of the individual cell can be adjusted to a desired degree.

Users can frequently self diagnose the portion of their back and/or spine which is causing pain and/or discomfort. The user can adjust the location/orientation of the manipulation device (for instance adjusting the alignment cell 28′ to the desired (or optimal) height), at which point, under user's own weight, the vertebrae is gently directed back into alignment.

In the embodiment shown, the manipulation device 19 further comprises an inflator for inflating said bladders selectively with a pressurized fluid. For instance, the inflator could be an air pump for filling the bladders with air, a water pump for filling the bladders with liquid, etc. The preferred fluid being air and the preferred inflator being an air pump. It is preferred that the bladders be configured for being selectively inflated to varying pressures (as desired) thereby creating a plurality of possible rigidities, from soft to firm. For instance, a user may desire to keep all of the bladders 28 generally inflated (soft), while over-inflating (firm)a bladder 28′, thereby using the over-inflated bladder 28′ to put particular pressure upon one or more out of alignment vertebrae 40.

Oriented along this medial axis is at least one muscle relaxation portion 18, a dedicated area of the device's surface that comes into contact with the muscles that surround the spinal column, providing muscle relaxation through a combination of massage, vibration, and/or heat therapy.

This embodiment further preferably comprising a heating means positioned in the body portion, the purpose of the heating means being to apply heat to the back muscles. By warming the muscles of the user's back, the muscles relax, thereby increasing the ability of the present invention to manipulate the user's spine back into proper alignment. It is preferred that the heating means comprise a plurality of heating coils in said body portion.

This embodiment further preferably comprising a vibrating means positioned in the body portion for vibrating the user's back, either directly or through contact with the manipulation device. The purpose of the vibrating means being to apply vibration to the back muscles. By vibrating the muscles of the users back, the muscles relax, thereby increasing the ability of the present invention to manipulate the user's spine back into proper alignment. This vibrating means may be used in conjunction with or may also comprise massage means for massaging (with or without vibration) the back of the user.

This embodiment further preferably comprising a power supply, preferably having a cord attached thereto for selectively connecting to an electrical outlet. This power supply could be coupled to said heating means for supplying power to the heating means. This power supply could also be coupled to the vibrating means for supplying power to the vibrating means. This power supply could also be coupled to the inflator for supplying power to the inflator. The power supply could comprise an AC connection, a DC connection, at least one disposable battery, at least one rechargeable battery, or other power sources/supplies.

Operation of this power supply and/or the various mechanical/electrical components (heating means, inflator (or other manipulation device), means for massage, vibrating means) of the present invention is accomplished (in this embodiment) through use of a control portion. This control portion (remote control, controller) can be integrated (for instance in a side panel of the body portion), or can be remote (wired or wireless). The control allows the user to operate the manipulation device, for instance increasing the height of one or more of the individual “alignment cells” (bladders) so as to apply increased pressure upon select areas of the spine. The control portion likewise can control the massage/vibration/heat features of the present invention (if present) used to achieve muscle relaxation. In the embodiment show, the control portion 30 is remote and wired, having buttons for controlling massage 20, vibration 22, and heat 26, as well as one or more buttons for controlling direction 29 and/or operation of the manipulation device 19.

Referring now to FIGS. 4-6, shown is a second embodiment of the present invention. This embodiment comprising a body portion 111 (pillow) contoured to generally correspond to the natural curve (lower, middle, and upper areas of the spine (lumbar, thoracic, cervical) as well as the head and neck) of a human spine 136. The body portion 111 defining a medial axis (generally along which the cross-section of FIG. 5 is taken) which is configured for generally aligning parallel to the spine of the user in use.

In preferred use, a user would place the device 110 upon a surface (preferably firm), such as the floor or a bed, and would lay supine upon the device, generally aligning his/her spine with the contours of the body portion 111. It is preferred that the body portion be cushioned, having a solid (firm) bottom portion (base unit) 114, a soft contoured top 116, and being covered with a cover or covering.

This embodiment showing a mechanical manipulation device 119 for selectively applying pressure against a selected region of the user's spine. This manipulation device selectively applying pressure to the spinal column, preferably on a particular vertebrae and/or the vertebrae adjacent thereto, thereby assisting in manipulating the spine back into correct alignment.

In the embodiment shown, the manipulation device 119 comprises at least one cam-shaped knob 128 able to rotate generally perpendicular to the medial axis to apply selective degrees of pressure upon a user's spine. The knob 128 could comprise other shapes, styles and configurations. The knob 128 being able to be moved along the medial axis to allow a user to provide pressure generally anywhere along the spine as desired, and varying the degree of rotation of the cam-shaped knob 128 results in varying degrees of pressure able to be applied to the spinal column.

There are countless ways a mechanical manipulation device could work. For instance, in one mechanical embodiment, a manipulation device is manually moved into various positions along the spine; at which point, its height can be adjusted to achieve the desired or optimal amount of pressure. Another mechanical embodiment might utilize a manipulation device that is moved into various positions along the spine through use of a motor and screw drive mechanism; at which point, its height can be adjusted to achieve the desired (or optimal) amount of pressure. Another mechanical embodiment might utilize a manipulation device that is moved into various positions along the spine via motor (or manual) and track; at which point, its height can be adjusted to achieve the desired (or optimal) amount of pressure.

Users can frequently self diagnose the portion of their back and/or spine which is causing pain and/or discomfort. The user can adjust the location/orientation of the manipulation device (for instance adjusting the knob 128 to the desired (or optimal) height), at which point, under user's own weight, the vertebrae is gently directed back into alignment.

In this particular embodiment, the manipulation device 119 would further comprise a motor (as part of a knob mechanism) for rotating the knob 128. Alternatively, the knob could be rotated by hand through use of a separate mechanical structure.

Oriented along this medial axis is a muscle relaxation portion 118, a dedicated area(s) of the device's surface that comes into contact with the muscles that surround the spinal column, providing muscle relaxation through a combination of massage, vibration, and/or heat therapy.

This embodiment further preferably comprising a heating means positioned in the body portion, the purpose of the heating means being to apply heat to the back muscles. By warming the muscles of the user's back, the muscles relax, thereby increasing the ability of the present invention to manipulate the user's spine back into proper alignment. It is preferred that the heating means comprise a plurality of heating coils in said body portion.

This embodiment further preferably comprising a vibrating means positioned in the body portion for vibrating the user's back, either directly or through contact with the manipulation device. The purpose of the vibrating means being to apply vibration to the back muscles. By vibrating the muscles of the users back, the muscles relax, thereby increasing the ability of the present invention to manipulate the user's spine back into proper alignment. This vibrating means may be used in conjunction with or may also comprise massage means for massaging (with or without vibration) the back of the user.

This embodiment further preferably comprising a power supply, preferably having a cord attached thereto for selectively connecting to an electrical outlet. This power supply could be coupled to said heating means for supplying power to the heating means. This power supply could also be coupled to the vibrating means for supplying power to the vibrating means. This power supply cold also be coupled to the knob mechanism for supplying power to the knob mechanism. The power supply could comprise an AC connection, a DC connection, at least one a disposable battery, at least one a rechargeable battery, or other power sources/supplies.

Operation of this power supply and/or the various mechanical/electrical components (heating means, knob mechanism (or other manipulation device), means for massage, vibrating means) of the present invention is accomplished (in this embodiment) through use of a control portion. This control portion (remote control, controller) can be integrated (for instance in a side panel of the body portion), or can be remote (wired or wireless). The control portion allows the user to operate the manipulation device, for instance, rotating the knob to increasing the height knob's end, thereby applying increased pressure upon select areas of the spine. The control portion likewise can control the massage/vibration/heat features of the present invention (if present) used to achieve muscle relaxation. In the embodiment show, the control portion 130 is remote and wired, having buttons for controlling massage 120, vibration 122, and heat 126, as well as one or more buttons for controlling direction 128 and/or operation of the manipulation device 119.

It is through the cooperation and use of one or more of: the pressure exerted by the manipulation device, the warmth from heating means, massaging from the means for massaging and/or the vibrations from said vibrating means that the user's spine is manipulated into proper alignment and/or pain/discomfort is reduced.

As discussed above, the manipulation device is intended to provide a means by which to target the various areas of the spine and apply pressure to the vertebrae(s) requiring re-alignment. This function could be performed by a number of alternative methods (i.e., mechanical, pneumatic, hydraulic, electro-mechanical, etc.). For instance, FIGS. 4-6 (described infra) show the utilization of a mechanical manipulation device, whereas FIGS. 1-3 show the utilization of a pneumatic or hydraulic manipulation device. In one electromechanical embodiment, a manipulation device is moved into various positions along the spine via a rail(s) and a series of magnets, at which point, its height can be adjusted to achieve the desired (or optimal) amount of pressure. Whatever manipulation device (mechanical or electromechanical) used, it can be rotated in place or simply raised/lowered through any number of manual or mechanical means.

The embodiment shown in the figures shows a “one size fits all” style of body portion. However, the body portion could be scalable, or configured for lengthening/shortening thereby allowing users to customize the body portion to their needs and/or frames. Also, while the present invention is envisioned as generally contained within the body portion, such components could be located external there-to. In another embodiment, the present invention is, rather than being built within a separate body portion, is configured for building into a chair, office furniture, automobile seats, chiropractor tables, beds, etc. The present invention also can be scaled down (for instance, providing only for the targeting of the upper back, or the lower back, etc.).

Additional possible features that could be included include: providing a speaker within the body portion for interfacing with an audio source (.mp3 player, compact disc player, etc), and the utilization of the device with relaxing audio (i.e., hypnosis, subliminal messaging, music, sounds of nature etc.) and/or aroma therapy to help the user relax (thereby allowing the present invention to work to manipulate the spine).

The purpose of the Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other features and advantages of the present invention will become readily apparent to those skilled in this art from the following detailed description describing preferred embodiments of the invention, simply by way of illustration of the best mode contemplated by carrying out our invention. As will be realized, the invention is capable of modification in various obvious respects all without departing from the invention. Accordingly, the drawings and description of the preferred embodiments are to be regarded as illustrative in nature, and not as restrictive in nature.

What is claimed is:

1. A device for manipulating the spine, comprising:
a body portion, said body portion contoured to correspond to the natural lumbar curve of a human spine, said body portion configured for being laid upon by a supine user, said body portion defining a medial axis which is configured for generally aligning with the spine area of said supine user;
a manipulation device for selectively applying pressure against a selected region of the user's spine, thereby pressuring said spine back into correct alignment, said manipulation device located along said medial axis;
a heating means positioned in said body portion for warming said spine area;
a vibrating means positioned in said body portion for vibrating said spine area;
a power supply, said power supply being coupled to said heating means for supplying power to said heating means, said power supply being coupled to said vibrating means for supplying power to said vibrating means; and
a control portion for controlling operation of said vibrating means and said heating means;
whereby the pressure from the manipulation device, the warmth from said heating means and the vibrations from said vibrating means cooperate to manipulate the user's spine into proper alignment.

2. The device of claim 1, wherein said manipulation device comprises a plurality of selectively inflatable bladders.

3. The device of claim 2, wherein said manipulation device comprises an inflator, said inflator configured for inflating said bladders selectively with a pressurized fluid.

4. The device of claim 3 wherein said pressurized fluid is air.

5. The device of claim 3, wherein said power supply is coupled to said inflator for supplying power to said inflator.

6. The device of claim 3, wherein said control portion controls operation of said inflator.

7. The device of claim 2, wherein said bladders can be selectively inflated to varying pressures thereby creating a plurality of be rigidities, from soft to firm.

8. The device of claim 2, wherein said body portion is a pillow.

9. The device of claim 1, wherein said power supply has a cord attached thereto for selectively connecting to an electrical outlet.

10. The device of claim 1, wherein said heating means comprises a plurality of heating coils in said body portion.

11. A device for manipulating the spine, comprising:
a body portion, said body portion contoured to correspond to the natural curve of a human spine, said body portion configured for being laid upon by a supine user, said body portion defining a medial axis which is configured for generally aligning with the spine area of said supine user;
a manipulation device for selectively applying pressure against a selected region of the user's spine, thereby pressuring said spine back into correct alignment, said manipulation device located along said medial axis, said manipulation device comprising a plurality of selectively inflatable bladders and an inflator configured for inflating said bladders selectively with air, wherein said bladders can be selectively inflated to varying pressures thereby creating a plurality of be rigidities, from soft to firm;
a heating means positioned in said body portion for warming said spine area;
a vibrating means positioned in said body portion for vibrating said spine area;
a power supply, said power supply being coupled to said heating means for supplying power to said heating means, said power supply being coupled to said vibrating means for supplying power to said vibrating means, said power supply being coupled to said inflator for supplying power to said inflator; and
a control portion for controlling operation of said vibrating means, said heating means and said inflator;
whereby the pressure from the manipulation device, the warmth from said heating means and the vibrations from said vibrating means cooperate to manipulate the user's spine into proper alignment.

12. The device of claim 11, wherein said body portion is a pillow.

13. The device of claim 11, wherein said power supply has a cord attached thereto for selectively connecting to an electrical outlet.

14. The device of claim 11, wherein said heating means comprises a plurality of heating coils in said body portion.

15. A device for manipulating the spine, comprising:
a body portion, said body portion contoured to correspond to the natural lumbar curve of a human spine, said body portion configured for being laid upon by a supine user, said body portion defining a medial axis which is configured for generally aligning with the spine area of said supine user;
a manipulation device for selectively applying pressure against a selected region of the user's spine, thereby pressuring said spine back into correct alignment, said manipulation device located along said medial axis and able to be moved along said medial axis, said manipulation device comprising a knob mechanism having a knob configured for selectively extending away from said body portion thereby applying a desired amount of pressure to said spine;
a heating means positioned in said body portion for warming said spine area;
a vibrating means positioned in said body portion for vibrating said spine area;
a power supply, said power supply being coupled to said heating means for supplying power to said heating means, said power supply being coupled to said vibrating means for supplying power to said vibrating means; and
a control portion for controlling operation of said vibrating means and said heating means;
whereby the pressure from the manipulation device, the warmth from said heating means and the vibrations from said vibrating means cooperate to manipulate the user's spine into proper alignment.

16. The device of claim 15, wherein said knob is generally cam-shaped and is able to rotate generally perpendicular to the medial axis thereby providing the selective extension.

17. The device of claim 15, wherein said power supply is coupled to said knob mechanism for supplying power to said knob mechanism for rotating said knob.

18. The device of claim 15, wherein said control portion is also for controlling operation of said knob mechanism.

19. The device of claim 15, wherein said power supply has a cord attached thereto for selectively connecting to an electrical outlet.

20. The device of claim 15, wherein said heating means comprises a plurality of heating coils in said body portion.
